(19) United States
(12) Patent Application Publication
(10) Pub. No.: US 2025/0195697 A1
(43) Pub. Date: Jun. 19, 2025

(54) RAPIDLY CROSSLINKABLE HYDROGELS WITH ENHANCED RADIOPACITY FOR MEDICAL APPLICATIONS

(71) Applicant: Boston Scientific Scimed, Inc., Maple Grove, MN (US)

(72) Inventors: Yen-Hao Hsu, Shrewsbury, MA (US); Joseph Thomas Delaney, JR., Minneapolis, MN (US); Cristian Parisi, Boston, MA (US); Nicolas Ball-Jones, Minneapolis, MN (US)

(73) Assignee: Boston Scientific Scimed, Inc., Maple Grove, MN (US)

(21) Appl. No.: 18/979,225

(22) Filed: Dec. 12, 2024

Related U.S. Application Data
(60) Provisional application No. 63/610,099, filed on Dec. 14, 2023.

(51) Int. Cl. A61K 49/04; A61M 5/31; A61M 5/315; C08G 63/133; C08J 3/075

(52) U.S. Cl. CPC A61K 49/0442; A61K 49/0457; A61M 5/3129; A61M 5/31596; C08G 63/133; C08J 3/075; C08J 2371/02; C08L 2312/00

(57) ABSTRACT

In some aspects, the present disclosure pertains to systems for forming crosslinked reaction products that comprises (a) a first composition that comprises a first multifunctional molecule that comprises a plurality of first reactive moieties that comprise azide groups and (b) a second composition that comprises a second multifunctional molecule that comprises a plurality of second reactive moieties that comprise cyclic alkyne groups, where at least one of the first and second multifunctional molecules is a reactive multi-arm polymer and where at least one of the first and second multifunctional molecules comprises an iodinated core. Other aspects of the present disclosure include crosslinked networks formed from such first and second multifunctional molecules and methods of treatment based on crosslinked networks formed from such first and second multifunctional molecules.

### CROSS-REFERENCE TO RELATED APPLICATIONS

This application claims the benefit of U.S. Provisional Patent Application Ser. No. 63/610,099 filed on Dec. 14, 2023, the disclosure of which is incorporated herein by reference.

### FIELD

The present disclosure relates hydrolysable hydrogels and to crosslinkable systems for forming hydrolysable hydrogels, among other aspects. The hydrogels and crosslinkable systems for forming the same are useful, for example, in various medical applications.

### BACKGROUND

SpaceOAR®, a rapid crosslinking hydrogel that polymerizes in vivo within seconds, is based on a multi-arm polyethylene glycol (PEG) polymer functionalized with succinimidyl glutarate as reactive end groups which further react with trilysine to form crosslinks. This product has become a very successful, clinically-used biomaterial in prostate cancer therapy. A further improvement based on this structure is that a portion the succinimidyl glutarate end groups have been functionalized with 2,3,5-triiiodobenzamide groups, providing radiopacity. This hydrogel, known by the trade name of SpaceOAR® Vue, is the radiopaque version of SpaceOAR® for prostate medical applications. Above a specific pH, the succinimidyl glutarate groups will rapidly react with the trilysine crosslinker in vivo to form a hydrogel. The hydrogels breakdown in-vivo over the course of ca. 6-9 months. The breakdown occurs primarily through the hydrolysis of the ester linkages associated with the glutarate groups.

While the above approach is effectual, some issues arise as a result of incorporation of the 2,3,5-triiiodobenzamide groups. First, in order to functionalize TIB on 8-arm PEG, the binding site from succinimidyl glutarate (SG) must be sacrificed for each functionalized arm, which can lead to longer gel times. Moreover, the entire functionalization process involves multiple steps, typically five steps, from commercially available hydroxyl-terminated 8-arm PEG to its functionalized form with two different end groups (TIB and SG groups). This complex process of synthesizing the 8-arm PEG results in a significant increase of the product cost, and more complex product quality control activities. In addition, the trilysine utilized as a crosslinker to form crosslinked hydrogel with multi-arm PEG needs to be stabilized in buffer solution with a controlled pH value in order to prevent crosslinking before injection introducing further complexity.

### SUMMARY

The present disclosure provides an alternative approach to the SpaceOAR® products described above.

In some aspects, the present disclosure pertains to systems for forming crosslinked reaction products that comprises (a) a first composition that comprises a first multifunctional molecule that comprises a plurality of first reactive moieties that comprise azide groups and (b) a second composition that comprises a second multifunctional molecule that comprises a plurality of second reactive moieties that comprise cyclic alkyne groups, where at least one of the first and second multifunctional molecules is a reactive multi-arm polymer and where at least one of the first and second multifunctional molecules comprises an iodinated core.

In some embodiments, the first reactive moieties further comprise hydrolysable linkages through which the azide groups are linked to a remainder of the first multifunctional molecule.

In some embodiments, which can be used in conjunction with the above aspects and embodiments, the cyclic alkyne groups are selected from cyclooctyne-containing groups, such as dibenzocyclooctyne groups, bicyclo[6.1.0]nonyne (BCN) groups, bicyclo[6.1.0]nonyne groups, difluorocyclooctyne groups, cyclooctyne groups, pyrrolocyclooctyne groups, and dibenzocyclooctyne groups.

In some embodiments, which can be used in conjunction with the above aspects and embodiments, the second reactive moieties further comprise hydrolysable linkages through which the cyclic alkyne groups are linked to a remainder of the first multifunctional molecule.

In some embodiments, which can be used in conjunction with the above aspects and embodiments, (a) the first multifunctional molecule is a multi-arm polymer having an iodinated core and a plurality of polymer arms that have azide end groups and (b) the second multifunctional molecule is a multi-arm polymer having a non-iodinated core and a plurality of polymer arms that have cyclic alkyne end groups, a multi-arm polymer having an iodinated core and a plurality of polymer arms that have cyclic alkyne end groups, a non-polymeric molecule having a non-iodinated core and a plurality of pendant reactive moieties that comprise cyclic alkyne groups, or a non-polymeric molecule having an iodinated core and a plurality of pendant reactive moieties that comprise cyclic alkyne groups.

In some embodiments, which can be used in conjunction with the above aspects and embodiments, (a) the first multifunctional molecule is a multi-arm polymer having a non-iodinated core and a plurality of polymer arms that have azide end groups and (b) the second multifunctional molecule is a multi-arm polymer having an iodinated core and a plurality of polymer arms that have cyclic alkyne end groups or a non-polymeric molecule having an iodinated core and a plurality of pendant reactive moieties that comprise cyclic alkyne groups.

In some embodiments, which can be used in conjunction with the above aspects and embodiments, (a) the first multifunctional molecule is a non-polymeric molecule having an iodinated core and a plurality of pendant reactive moieties that comprise azide groups and (b) the second multifunctional molecule is a multi-arm polymer having a non-iodinated core and a plurality of polymer arms that have cyclic alkyne end groups or a multi-arm polymer having an iodinated core and a plurality of polymer arms that have cyclic alkyne end groups.

In some embodiments, which can be used in conjunction with the above aspects and embodiments, (a) the first multifunctional molecule is a non-polymeric molecule having a non-iodinated core and a plurality of pendant reactive moieties that comprise azide groups and (b) the second multifunctional molecule is a multi-arm polymer having an iodinated core and a plurality of polymer arms that have cyclic alkyne end groups.

In some embodiments, which can be used in conjunction with the above aspects and embodiments, the iodinated core comprises one or more iodinated aromatic groups.

In some embodiments, which can be used in conjunction with the above aspects and embodiments, the iodinated core comprises an iodinated polyol residue.

In some embodiments, which can be used in conjunction with the above aspects and embodiments, the first composition is provided in a first container and the second composition is provided in a second container. In some of these embodiments, the first and second containers are independently selected from vials and syringes.

In some embodiments, which can be used in conjunction with the above aspects and embodiments, the system further comprises a delivery device that is configured to deliver the first and second compositions. In some of these embodiments, the delivery device comprises a dual barrel syringe.

In other aspects, the present disclosure pertains to a crosslinked network formed by combining (a) a first multifunctional molecule in that comprises a plurality of first reactive moieties that comprise azide groups and (b) a second multifunctional molecule that comprises a plurality of second reactive moieties that comprise cyclic alkyne groups, wherein at least one of the first and second multifunctional molecules is a reactive multi-arm polymer and wherein at least one of the first and second multifunctional molecules comprises an iodinated core. The first and second multifunctional molecules may be selected, for example, from those of any of the preceding embodiments.

In some embodiments, the crosslinked network contains is a hydrogel.

In some embodiments, which can be used in conjunction with the above aspects and embodiments, the crosslinked network has a radiopacity that is greater than 250 Hounsfield units.

In some aspects, the present disclosure provides a suspension of particles of a crosslinked network in accordance with any of the above aspects and embodiments. In some cases, the suspension of particles may be provided in a syringe.

In other aspects, the present disclosure provides a method of treatment comprising administering to a subject a mixture that comprises (a) a first multifunctional molecule that comprises a plurality of first reactive moieties that comprise azide groups and (b) a second multifunctional molecule that comprises a plurality of second reactive moieties that comprise cyclic alkyne groups, wherein at least one of the first and second multifunctional molecules is a reactive multi-arm polymer and wherein at least one of the first and second multifunctional molecules comprises an iodinated core. The first and second multifunctional molecules may be selected, for example, from those of any of the preceding embodiments.

Potential benefits of the present disclosure include the following: high X-ray visibility, improved in vivo persistence, good homogeneity, and rapid and efficient hydrogel formation without the need or buffer solutions or catalysts.

The above and other aspects, embodiments, features and benefits of the present disclosure will be readily apparent from the following detailed description.

### BRIEF DESCRIPTION OF THE DRAWINGS

FIG. 1A schematically illustrates a method of forming hydroxyl-terminated multi-arm PEG, in accordance with an embodiment of the present disclosure.

FIG. 1B schematically illustrates a method of forming a dibenzocyclooctyne-terminated multi-arm PEG from the hydroxyl-terminated multi-arm PEG of FIG. 1A, in accordance with an embodiment of the present disclosure.

FIG. 2A schematically illustrates a method of forming iodinated hydroxyl-terminated multi-arm PEG, in accordance with an embodiment of the present disclosure.

FIG. 2B schematically illustrates a method of forming an iodinated azide-terminated multi-arm PEG from the iodinated hydroxyl-terminated multi-arm PEG of FIG. 2A, in accordance with an embodiment of the present disclosure.

FIG. 3 schematically illustrates an iodinated dibenzocyclooctyne-terminated multi-arm PEG in accordance with an embodiment of the present disclosure.

FIG. 4 schematically illustrates an azide-terminated multi-arm PEG, in accordance with an embodiment of the present disclosure.

FIG. 5 schematically illustrates the formation of a crosslinked product resulting from a strain-promoted alkyne-azide cycloaddition reaction between the dibenzocyclooctyne-terminated multi-arm PEG of FIG. 1B and the iodinated azide-terminated multi-arm PEG of FIG. 2B, in accordance with an embodiment of the present disclosure.

FIG. 6 schematically illustrates a delivery device, in accordance with an embodiment of the present disclosure.

FIG. 7 schematically illustrates a delivery device, in accordance with another embodiment of the present disclosure.

### DETAILED DESCRIPTION

In some aspects, the present disclosure provides systems that comprise (a) a first multifunctional molecule that comprises a plurality of first reactive moieties that comprise azide (—N₃) groups and (b) a second multifunctional molecule that comprises a plurality of second reactive moieties that comprise cyclic alkyne groups, wherein at least one of the multifunctional molecules is a multi-arm polymer and wherein at least one of the multifunctional molecules comprises an iodinated core.

The azide groups of the first multifunctional molecule and the strained cyclic alkyne end groups of the second multifunctional molecule can react via a strain-promoted alkyne/azide cycloaddition (SPAAC) reaction which is an “ultra-fast” click reaction. In this way, such systems can be used to form radiopaque crosslinked networks, including radiopaque hydrogels. As used herein, a “hydrogel” is a crosslinked polymer that can absorb water but does not dissolve when placed in water.

In some embodiments, the first reactive moieties comprise azide groups further comprise a hydrolysable linkage, such as a hydrolysable ester group or a carbonate linkage, through which the azide groups are linked to a remainder of the first multifunctional molecule.

Examples of second reactive moieties that comprise cyclic alkyne groups include reactive moieties that comprise a strained cyclic alkyne group, for instance, a cyclooctyne-containing group, such as a dibenzocyclooctyne group, for example, a

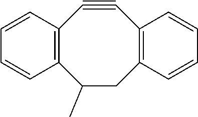 [SMILES: CC1Cc2ccccc2C#Cc2ccccc21]

group or a

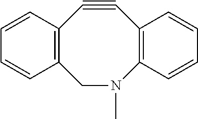 [SMILES: CN1Cc2ccccc2C#Cc2ccccc21]

group, a bicyclo[6.1.0]nonyne (BCN) group,

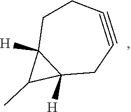

a difluorocyclooctyne (DIFO) group,

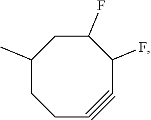

a cyclooctyne (OCT) group,

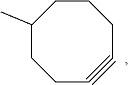

or a pyrrolocyclooctyne group,

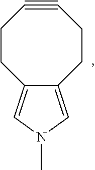

among others.

In some embodiments, the second reactive moieties further comprise a hydrolysable linkage, such as a hydrolysable ester group or a carbonate group, through which the cyclic alkyne groups are linked to a remainder of the second multifunctional molecule. Examples of such second reactive moieties include, for example, an 11,12-didehydro-oxodibenz[b,f]azocine-5(6H)-alkanoic acid ester group, more specifically, an 11,12-didehydro-oxodibenz[b,f]azocine-5(6H)—C₃-C₁₀-alkanoic acid ester group, such as an 11,12-didehydro-T-oxodibenz[b,f]azocine-5(6H)-butanoic acid ester group,

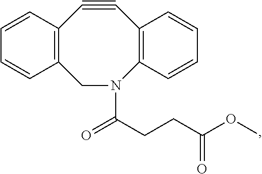

also known as a DBCO acid ester group, an 11,12-didehydro-δ-oxodibenz[b,f]azocine-5(6H)-pentanoic acid ester group, an 11,12-didehydro-ε-oxodibenz[b,f]azocine-5(6H)-hexanoic acid ester group. Further examples include other cyclooctyne-containing acid ester groups, such as bicyclo[6.1.0]nonyne acid ester groups, difluorocyclooctyne acid ester groups, cyclooctyne acid ester groups, pyrrolocyclooctyne acid ester groups, and dibenzocyclooctyne acid ester groups, among others.

In some embodiments, the first reactive moieties and/or the second reactive moieties further comprise iodine groups.

Reactive multi-arm polymers for use in conjunction with the present disclosure include reactive multi-arm polymers having polymer arms that comprise (a) a polymer segment linked to a core region and (b) a terminal reactive moiety selected from either a first reactive moiety that comprise an azide group or a second reactive moiety that comprises a cyclic alkyne group. In some embodiments, the core region comprises a polyol residue. In some embodiments, the core region comprises an iodinated polyol residue.

Polymer segments for the polymer arms may be selected from any of a variety of synthetic, natural, or hybrid synthetic-natural polymer segments. Examples of polymer segments include those that are formed from one or more monomers selected from the following: C₁-C₆-alkylene oxides (e.g., ethylene oxide, propylene oxide, tetramethylene oxide, etc.), cyclic ester monomers (e.g. glycolide, lactide, β-propiolactone, β-butyrolactone, γ-butyrolactone, γ-valerolactone, δ-valerolactone, ε-caprolactone, etc.), oxazoline monomers (e.g., oxazoline and 2-alkyl-2-oxazolines, for instance, 2-(C₁-C₆ alkyl)-2-oxazolines, including various isomers, such as 2-methyl-2-oxazoline, 2-ethyl-2-oxazoline, 2-n-propyl-2-oxazoline, 2-isopropyl-2-oxazoline, 2-n-butyl-2-oxazoline, 2-isobutyl-2-oxazoline, 2-hexyl-2-oxazoline, etc.), 2-phenyl-2-oxazoline, polar aprotic vinyl monomers (e.g. N-vinyl pyrrolidone, acrylamide, N-methyl acrylamide, dimethyl acrylamide, N-vinylimidazole, 4-vinylimidazole, sodium 4-vinylbenzenesulfonate, etc.), dioxanone, N-isopropylacrylamide, amino acids and sugars.

Polymer segments for the polymer arms may be selected, for example, from the following polymer segments: polyether segments including poly(C₁-C₆-alkylene oxide) segments such as poly(ethylene oxide) (PEO) segments (also referred to as polyethylene glycol segments or PEG segments), poly(propylene oxide) segments, poly(ethylene oxide-co-propylene oxide) segments, polyester segments including polyglycolide segments, polylactide segments, poly(lactide-co-glycolide) segments, poly(O-propiolactone) segments, poly(β-butyrolactone) segments, poly(γ-butyrolactone) segments, poly(γ-valerolactone) segments, poly(δ-valerolactone) segments, and poly(ε-caprolactone) segments, polyoxazoline segments including poly(2-C₁-C₆-alkyl-2-oxazoline segments) such as poly(2-methyl-2-oxazoline) segments, poly(2-ethyl-2-oxazoline) segments, poly(2-propyl-2-oxazoline) segments, poly(2-isopropyl-2-oxazoline) segments, and poly(2-n-butyl-2-oxazoline) segments, poly(2-phenyl-2-oxazoline) segments, polymer segments formed from one or more polar aprotic vinyl monomers, including poly(N-vinyl pyrrolidone) segments, poly(acrylamide) segments, poly(N-methyl acrylamide) segments, poly(dimethyl acrylamide) segments, poly(N-vinylimidazole) segments, poly(4-vinylimidazole) segments, and poly(sodium 4-vinylbenzenesulfonate) segments, polydioxanone segments, poly(N-isopropylacrylamide) segments, polypeptide segments, and polysaccharide segments.

Polymer segments for use in the multi-arm polymers of the present disclosure typically contain between 2 and 1000 monomer units or more, for example, ranging anywhere from 2 to 5 to 10 to 25 to 50 to 100 to 250 to 500 to 1000 monomer units (i.e., ranging between any two of the preceding values). In other cases, the polymer segments of the present disclosure can be replaced by a single monomer unit.

Polyol residues in accordance with the present disclosure include residues of polyols having three more hydroxyl groups, for example, containing anywhere from 3 to 100 hydroxyl groups (e.g., having 3, 4, 5, 6, 7, 8, 9, 10, 12, 15, 20, 25, 30, 40, 50, 60, 70, 80, 90 or 100 hydroxyl groups). General classes of polyols include sugars (monosaccharides, disaccharides, trisaccharides, etc.), sugar alcohols, calixarenes, polyhedral oligomeric silsesquioxanes (POSS), cyclodextrin, polyhydroxylated polymers, catechins, flavanols, anthocyanins, stilbenes, and polyphenols, among others.

Illustrative polyols may be selected, for example, from straight-chained, branched and cyclic aliphatic polyols including straight-chained, branched and cyclic polyhydroxyalkanes, straight-chained, branched and cyclic polyhydroxy ethers, including polyhydroxy polyethers, straight-chained, branched and cyclic polyhydroxyalkyl ethers, including polyhydroxyalkyl polyethers, straight-chained, branched and cyclic sugars and sugar alcohols, such as glycerol, mannitol, sorbitol, inositol, xylitol, quebrachitol, threitol, arabitol, erythritol, pentaerythritol, dipentaerythritol, tripentaerythritol, adonitol, hexaglycerol, dulcitol, fucose, ribose, arabinose, xylose, lyxose, rhamnose, galactose, glucose, fructose, sorbose, mannose, pyranose, altrose, talose, tagatose, pyranosides, sucrose, lactose, and maltose, polymers (defined herein as two or more units) of straight-chained, branched and cyclic sugars and sugar alcohols, including oligomers (defined herein as ranging from two to ten units, including dimers, trimers, tetramers, pentamers, hexamers, heptamers, octamers, enneamers and decamers) of straight-chained, branched and cyclic sugars and sugar alcohols, including the preceding sugars and sugar alcohols, starches, amylose, dextrins, cyclodextrins, polyhedral oligomeric silsesquioxanes (POSS), catechins, flavanols, anthocyanins, stilbenes, polyphenols, as well as polyhydroxy crown ethers, and polyhydroxyalkyl crown ethers. Illustrative polyols also include aromatic polyols including 1,1,1-tris(4′-hydroxyphenyl) alkanes, such as 1,1,1-tris(4-hydroxyphenyl)ethane, and 2,6-bis(hydroxyalkyl)cresols, among others.

In some embodiments, iodinated polyols may be employed to provide the resulting multifunctional molecule with radiopacity. Iodinated polyols include iodinated aromatic polyols, examples of which are compounds that comprise 3 or more hydroxyl groups, and one or more iodinated aromatic groups. Examples of iodinated aromatic groups include iodine-substituted monocyclic aromatic groups and iodine-substituted multicyclic aromatic groups, such as iodine-substituted phenyl groups, iodine-substituted naphthyl groups, iodine-substituted anthracenyl groups, iodine-substituted phenanthrenyl groups and iodine-substituted tetracenyl groups, among others. The aromatic groups may be substituted with one, two, three, four, five, six or more iodine atoms. In various embodiments, the aromatic groups are further substituted with the three or more hydroxyl groups, which may be directly substituted to the aromatic groups or may be provided in the form of hydroxyalkyl groups (e.g., C₁-C₄-hydroxyalkyl groups containing one, two, three or four carbon atoms and containing one, two, three or four or more hydroxyl groups). The hydroxyalkyl groups may be linked to the aromatic group directly or through any suitable linking moiety, which may be selected, for example, from amide groups, amine groups, ether groups, ester groups, or carbonate groups, among others.

Specific examples of iodinated polyols for use in the present disclosure include iodinated polyols that are known for use as iodinated contrast agents, whose biocompatibility has been demonstrated to be reasonably well tolerated. Specific examples of iodinated polyols include commercially available 1,3,5 triiodo-2,4,6-trishydroxymethylbenzene (CAS #178814-33-0),

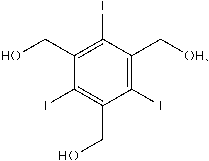 [SMILES: OCc1c(I)c(CO)c(I)c(CO)c1I]

iodixanol (CAS #92339-11-2),

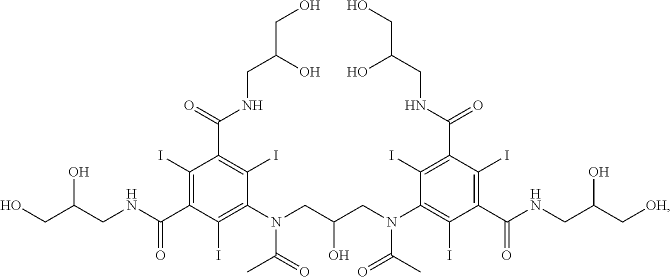 [SMILES: CC(=O)N(CC(O)CN(C(C)=O)c1c(I)c(C(=O)NCC(O)CO)c(I)c(C(=O)NCC(O)CO)c1I)c1c(I)c(C(=O)NCC(O)CO)c(I)c(C(=O)NCC(O)CO)c1I]

iotrolan (CAS #79770-24-4),

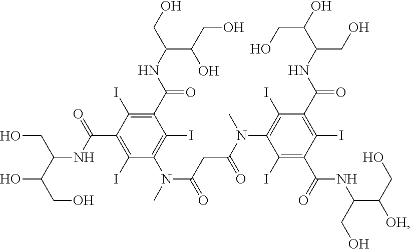 [SMILES: CN(C(=O)CC(=O)N(C)c1c(I)c(C(=O)NC(CO)C(O)CO)c(I)c(C(=O)NC(CO)C(O)CO)c1I)c1c(I)c(C(=O)NC(CO)C(O)CO)c(I)c(C(=O)NC(CO)C(O)CO)c1I]

iohexol (CAS #66108-95-0),

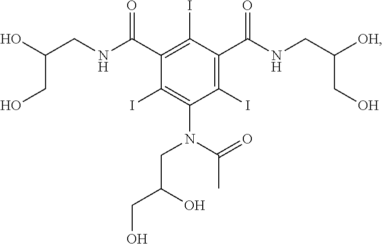 [SMILES: CC(=O)N(CC(O)CO)c1c(I)c(C(=O)NCC(O)CO)c(I)c(C(=O)NCC(O)CO)c1I]

ioversol (CAS #87771-40-2),

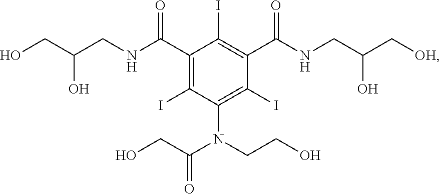 [SMILES: O=C(NCC(O)CO)c1c(I)c(C(=O)NCC(O)CO)c(I)c(N(CCO)C(=O)CO)c1I]

iopamidol (CAS #60166-93-0),

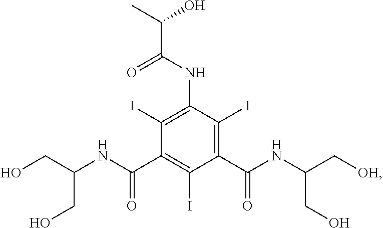 [SMILES: C[C@H](O)C(=O)Nc1c(I)c(C(=O)NC(CO)CO)c(I)c(C(=O)NC(CO)CO)c1I]

iohexol impurity J (CAS #76801-93-9),

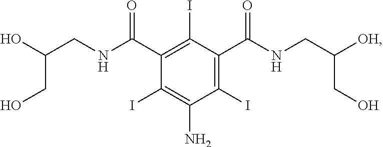 [SMILES: Nc1c(I)c(C(=O)NCC(O)CO)c(I)c(C(=O)NCC(O)CO)c1I]

and iopromide (CAS #73334-07-3),

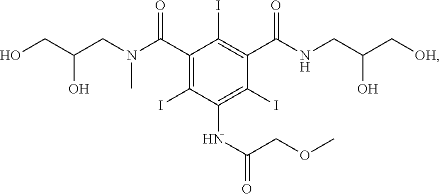 [SMILES: COCC(=O)Nc1c(I)c(C(=O)NCC(O)CO)c(I)c(C(=O)N(C)CC(O)CO)c1I]

among others.

In some embodiments, preformed polymer segments may be attached to the above polyols. For example, carboxyl terminated polymer segments may be attached to hydroxyl groups of the polyols through an ester coupling reaction.

In some embodiments of the present disclosure, non-iodinated or iodinated polyols such as those described above are used as multi-functional initiators for polymer chain growth, leading to hydroxy terminated multi-arm polymers. For example, the non-iodinated or iodinated polyols may be used as initiators for ring-opening polymerization of ethylene oxide to form poly(ethylene oxide) (PEG) segments at each of the hydroxyl groups of the polyol. The resulting hydroxy-terminated PEG segments possess tunable hydrophilicity depending on the desired water-solubility of the resulting multi-arm polymer, for example, with increasing PEG segment length leading to increasing hydrophilicity.

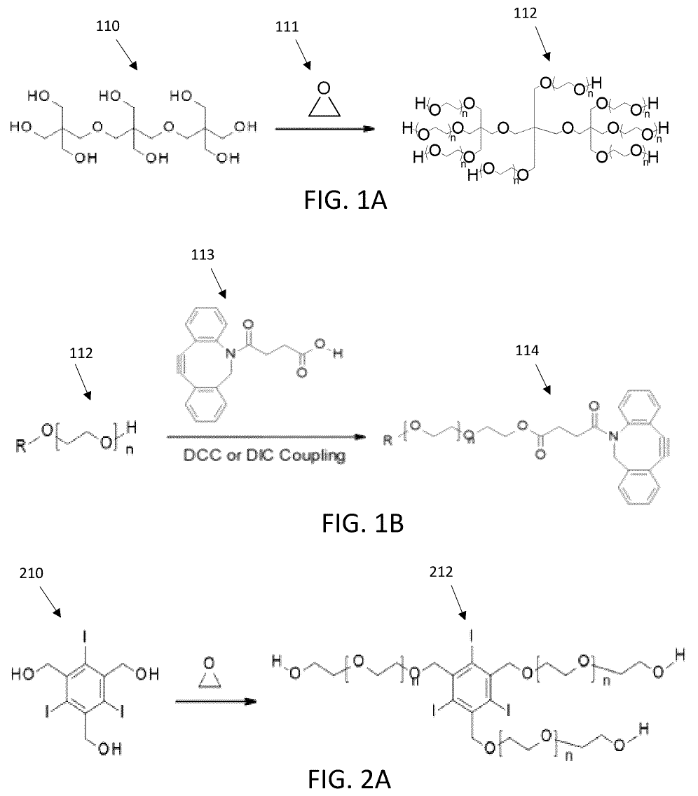

In a particular embodiment shown in FIG. 1A, commercially available tripentaerythritol (110) (CAS #78-24-0) can be used as an octa-functional initiator, which undergoes ring-opening polymerization with ethylene oxide (111). The polymerization process leads to poly(ethylene oxide) (PEG) chain growth at each of the eight hydroxyl groups of the tripentaerythritol, forming a hydroxy-terminated 8-arm-PEG (112) having a tripentaerythritol residue core. In FIG. 1A, n is an integer representing the number of monomer units in each polymer segment shown. In other cases n=1.

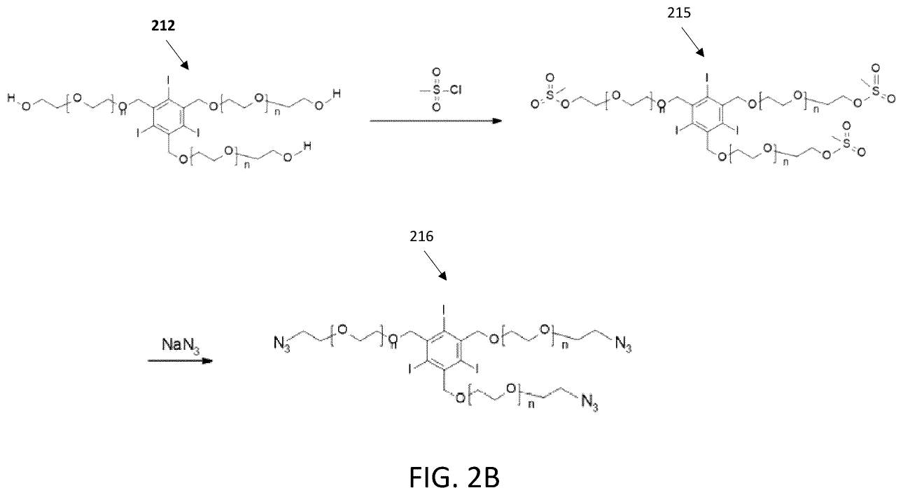

The strategy shown in FIG. 1A is broadly applicable and can be used in conjunction with a range of polyols, including those described above. In a particular embodiment shown in FIG. 2A, an iodinated polyol, 1,3,5-triiodo-2,4,6-tris-hydroxymethylbenzene (210), is used as an initiator, which undergoes ring-opening polymerization with ethylene oxide. The polymerization process leads to poly(ethylene oxide) (PEG) chain growth at each of the three hydroxyl groups of the 1,3,5-triiodo-2,4,6-tris-hydroxymethylbenzene (210). The resulting multi-arm polymer (212) contains three PEG arms that extend from a 1,3,5-triiodo-2,4,6-tris-hydroxymethylbenzene residue core. Each of the PEG arms has a terminal hydroxyl group. In FIG. 2A, n is an integer representing the number of monomer units in each polymer segment shown. In other cases n=1.

In some embodiments where an ester is desired as a hydrolysable linkage, terminal hydroxyl groups of hydroxy-terminated multi-arm polymers such as those produced in conjunction with FIG. 1A or FIG. 2A, among many others, may be reacted with a lactone (e.g., α-acetolactone, β-propiolactone, γ-butyrolactone, and δ-valerolactone, ε-caprolactone, etc.) to form a hydroxyl-terminated multi-arm polymer comprising a hydroxyl end group that is linked to a polymer segment through a hydrolysable ester group.

In other embodiments where an ester is desired as a hydrolysable linkage, terminal hydroxyl groups of hydroxy-terminated multi-arm polymers such as those produced in conjunction with FIG. 1A or FIG. 2A, among many others, may be reacted with a cyclic anhydride (e.g., glutaric anhydride, succinic anhydride, malonic anhydride, adipic anhydride, diglycolic anhydride, 1,3-acetonedicarboxylic acid anhydride, etc.) to form an acid-end-capped polymer segment such as a glutaric-acid-end-capped segment, a succinic-acid-end-capped segment, a malonic-acid-end-capped segment, an adipic-acid-end-capped segment, a diglycolic-acid-end-capped segment, a 1,3-acetonedicarboxylic-acid-end-capped segment, and so forth. The preceding cyclic anhydrides, among others, may be reacted with a hydroxy-terminated multi-arm polymer under basic conditions to form a carboxylic-acid-terminated multi-arm polymer comprising a carboxylic acid end group that is linked to a polymer segment through a hydrolysable ester group. Subsequently, the carboxylic acid end groups may be reduced to hydroxyl groups using a suitable reducing agent such as borane (BH₃).

Hydroxy-terminated multi-arm polymers such as those described above among many others, may be used to form multi-arm polymers that comprise a plurality of reactive moieties that comprise cyclic alkyne groups in accordance with the present disclosure and, in some embodiments, multi-arm polymers that comprise reactive moieties that comprise a cyclic alkyne groups and a hydrolysable ester group through which the cyclic alkyne groups are linked to a remainder of the multi-arm polymer.

For example, the carboxylic acid group of a molecule comprising a strained cyclic alkyne group and a carboxylic acid group may be reacted with hydroxyl groups of a hydroxy-terminated multi-arm polymer in an ester coupling reaction to form a strained-cyclic-alkyne-terminated multi-arm polymer. The ester coupling between the carboxylic acid and hydroxyl groups may be performed in the presence of a suitable coupling agent, for example, a carbodiimide coupling agent such as N,N′-dicyclohexylcarbodiimide (DCC) or N,N′-Diisopropylcarbodiimide (DIC). The ester linkage that is formed is hydrolysable. In this way a one-step functionalization strategy can be used to produce a strained-cyclic-alkyne-terminated multi-arm polymer from a hydroxy-terminated multi-arm polymer.

In a particular example shown in FIG. 1B, the hydroxy-terminated 8-arm-PEG (112) of FIG. 1A may be coupled with 11,12-didehydro-T-oxodibenz[b,f]azocine-5(6H)-butanoic acid (113) (CAS #1353016-70-2), also known as Dibenzocyclooctyne-acid or DBCO-acid, through DCC or DIC coupling to obtain a dibenzocyclooctyne-terminated 8-Arm-PEG polymer (114). In FIG. 1B, R represents a remainder of the molecule (including the tripentaerythritol residue core and the remaining 7 arms of the molecule).

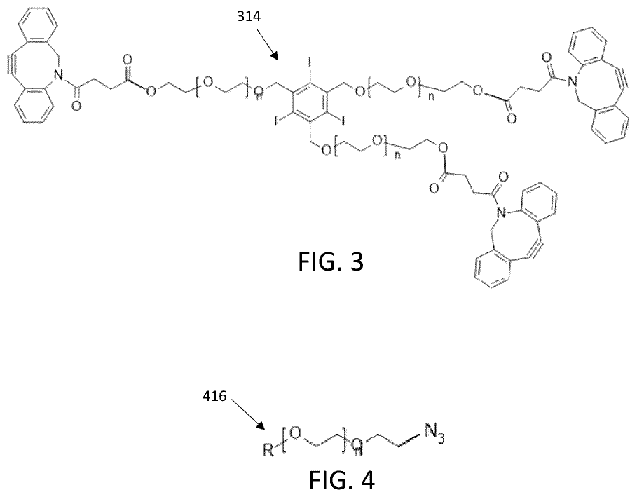

The strategy shown in FIG. 1B is widely applicable to other hydroxy-terminated polymers. For example, in another embodiment, the iodinated hydroxy-terminated 3-arm PEG of FIG. 2A may likewise be coupled with 11,12-Didehydro-T-oxodibenz[b,f]azocine-5(6H)-butanoic acid through DCC or DIC coupling to obtain an iodinated dibenzocyclooctyne-terminated 3-Arm-PEG polymer (314) as shown in FIG. 3.

In other embodiments, multifunctional molecules having strained cyclic alkyne groups in accordance with the present disclosure may be formed directedly from polyols, including non-iodinated or iodinated polyols such as those described above, without the formation of an intervening polymer arm. These resulting multifunctional molecules are generally non-polymeric in nature and comprise a non-iodinated or iodinated polyol residue core with multiple pendant strained cyclic alkyne groups.

For example, the carboxylic acid group of a molecule comprising a strained cyclic alkyne group and a carboxylic acid group may be reacted with the hydroxyl groups of a non-iodinated or iodinated polyol in an ester coupling reaction to form a multifunctional molecule that comprise a polyol residue with multiple strained cyclic alkyne pendant groups formed at the locations of the replaced/substituted hydroxyl groups. The ester coupling between the carboxylic acid and the hydroxyl groups may be performed in the presence of a suitable coupling agent, for example, a carbodiimide coupling agent such as DCC or DIC. The ester linkage that is formed may be subject to hydrolysis.

Hydroxy-terminated multi-arm polymers such as those described above may also be used to form multi-arm polymers that comprises a plurality of reactive moieties that comprise azide groups in accordance with the present disclosure, for example, multifunctional molecules that comprises a polyol residue core and a plurality PEG arms each terminated with an azide group, in accordance with the present disclosure.

In some embodiments, a hydroxy-terminated multi-arm polymer may first be reacted with methanesulfonyl chloride to covert the hydroxyl groups into methanesulfonate groups. The resulting methanesulfonate-terminated multi-arm polymer may then be reacted with sodium azide (NaN₃), which is used as a nucleophile to replace the methanesulfonate groups with azide groups. This two-step functionalization strategy results in an azide-terminated multi-arm polymer.

In a particular example shown in FIG. 2B, the iodinated hydroxy-terminated 3-arm PEG (212) of FIG. 2A is reacted with methanesulfonyl chloride (CAS #124-63-0) to obtain an iodinated methanesulfonyl-terminated 3-arm PEG (215). Sodium azide (NaN₃) is then reacted with the iodinated methanesulfonyl-terminated 3-arm PEG (215) to replace the methanesulfonate groups with azide groups, forming an iodinated azide-terminated 3-arm PEG (216).

The strategy shown in FIG. 2B is widely applicable to other hydroxy-terminated polymers. For example, in another embodiment, the hydroxy-terminated 8-arm PEG (112) of FIG. 1A may likewise be reacted with methanesulfonyl chloride, followed by sodium azide, to form an azide-terminated 8-arm-PEG polymer (416) as shown in FIG. 4.

In other embodiments, multifunctional molecules that comprise azide groups in accordance with the present disclosure are formed directedly from polyols, including non-iodinated or iodinated polyols such as those described above, without the formation of an intervening polymer arm. These resulting multifunctional molecules are thus generally non-polymeric in nature and comprise a non-iodinated or iodinated polyol residue core with multiple pendant azide groups.

For example, hydroxyl groups of the polyol may first be reacted with methanesulfonyl chloride to covert the hydroxyl groups into methanesulfonate groups, resulting in a multifunctional molecule that comprises a polyol residue with multiple pendant methanesulfonate groups. This molecule may then be reacted with sodium azide to replace the methanesulfonate groups with azide groups, forming a multifunctional molecule that comprises a polyol residue with pendant azide groups formed at the locations of the replaced/substituted hydroxyl groups.

The above and other techniques may be used to form the following: a first multifunctional molecule in the form of a multi-arm polymer having a non-iodinated core and a plurality of polymer arms that have azide end groups, a first multifunctional molecule in the form of a multi-arm polymer having an iodinated core and a plurality of polymer arms that have azide end groups, a first multifunctional molecule in the form of a non-polymeric molecule having a non-iodinated core and a plurality of pendant reactive moieties that comprise azide groups, a first multifunctional molecule in the form of a non-polymeric molecule having an iodinated core and a plurality of pendant reactive moieties that comprise azide groups, a second multifunctional molecule in the form of a multi-arm polymer having a non-iodinated core and a plurality of polymer arms that have cyclic alkyne end groups, a second multifunctional molecule in the form of a multi-arm polymer having an iodinated core and a plurality of polymer arms that have cyclic alkyne end groups, a second multifunctional molecule in the form of a non-polymeric molecule having a non-iodinated core and a plurality of pendant reactive moieties that comprise cyclic alkyne groups, and a second multifunctional molecule in the form of a non-polymeric molecule having an iodinated core and a plurality of pendant reactive moieties that comprise cyclic alkyne groups.

As noted above, in some aspects, the present disclosure provides systems that comprise (a) a first multifunctional molecule that comprises a plurality of reactive moieties that comprise azide groups and (b) a second multifunctional molecule that comprises a plurality of reactive moieties that comprise cyclic alkyne groups, wherein at least one of the first and second multifunctional molecules is a reactive multi-arm polymer and wherein at least one of the first and second multifunctional molecules comprises an iodinated core.

For example, the system may comprise (a) first multifunctional molecule in the form of a multi-arm polymer having a non-iodinated core and a plurality of polymer arms that have azide end groups and (b) a second multifunctional molecule in the form of a non-polymeric molecule having an iodinated core and a plurality of pendant reactive moieties that comprise cyclic alkyne groups.

As another example, the system may comprise (a) first multifunctional molecule in the form of a multi-arm polymer having a non-iodinated core and a plurality of polymer arms that have azide end groups and (b) a second multifunctional molecule in the form of a multi-arm polymer having an iodinated core and a plurality of polymer arms that have cyclic alkyne end groups.

As another example, the system may comprise (a) first multifunctional molecule in the form of a multi-arm polymer having an iodinated core and a plurality of polymer arms that have azide end groups and (b) a second multifunctional molecule in the form of a non-polymeric molecule having a non-iodinated core and a plurality of pendant reactive moieties that comprise cyclic alkyne groups.

As another example, the system may comprise (a) first multifunctional molecule in the form of a multi-arm polymer having an iodinated core and a plurality of polymer arms that have azide end groups and (b) a second multifunctional molecule in the form of a non-polymeric molecule having an iodinated core and a plurality of pendant reactive moieties that comprise cyclic alkyne groups.

As another example, the system may comprise (a) first multifunctional molecule in the form of a multi-arm polymer having an iodinated core and a plurality of polymer arms that have azide end groups and (b) a second multifunctional molecule in the form of a multi-arm polymer having a non-iodinated core and a plurality of polymer arms that have cyclic alkyne end groups.

As another example, the system may comprise (a) first multifunctional molecule in the form of a multi-arm polymer having an iodinated core and a plurality of polymer arms that have azide end groups and (b) a second multifunctional molecule in the form of a multi-arm polymer having an iodinated core and a plurality of polymer arms that have cyclic alkyne end groups.

As another example, the system may comprise (a) a first multifunctional molecule in the form of a non-polymeric molecule having a non-iodinated core and a plurality of pendant reactive moieties that comprise azide groups and (b) a second multifunctional molecule in the form of a multi-arm polymer having an iodinated core and a plurality of polymer arms that have cyclic alkyne end groups.

As another example, the system may comprise (a) a first multifunctional molecule in the form of a non-polymeric molecule having an iodinated core and a plurality of pendant reactive moieties that comprise azide groups and (b) a second multifunctional molecule in the form of a multi-arm polymer having a non-iodinated core and a plurality of polymer arms that have cyclic alkyne end groups.

As another example, the system may comprise (a) a first multifunctional molecule in the form of a non-polymeric molecule having an iodinated core and a plurality of pendant reactive moieties that comprise azide groups and (b) a second multifunctional molecule in the form of a multi-arm polymer having an iodinated core and a plurality of polymer arms that have cyclic alkyne end groups.

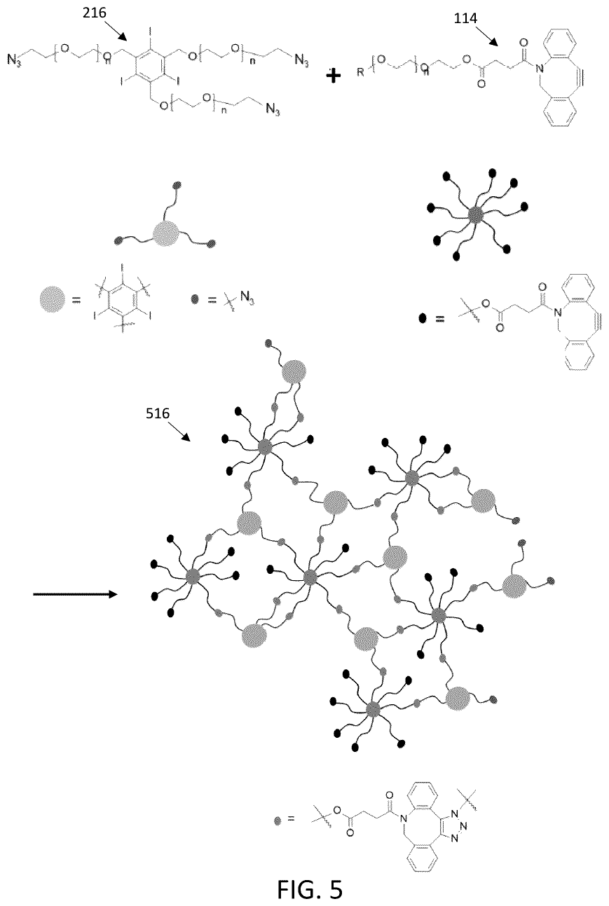

As also noted above, the azide groups of the first multifunctional molecule and the strained cyclic alkyne end groups of the second multifunctional molecule in such systems can react via a strain-promoted alkyne/azide cycloaddition to form radiopaque crosslinked reaction products, including radiopaque hydrogels. For example, in one particular embodiment shown in FIG. 5, the dibenzocyclooctyne-terminated multi-arm PEG (114) of FIG. 1B is reacted in a strain-promoted alkyne-azide cycloaddition reaction with the iodinated azide-terminated multi-arm PEG (216) of FIG. 2B to form a radiopaque crosslinked reaction product (516). Such crosslinking reactions occur spontaneously under mild conditions (e.g., in aqueous solutions at a pH ranging from 6 to 7) while producing no reaction by-products and rapidly reaching completion, for example, with crosslinking times ranging from 3 to 10 seconds. There is no need for buffer solutions or catalysts, reducing the complexity of the crosslinking systems.

In various embodiments, the radiopaque crosslinked reaction products (including the radiopaque hydrogels) have a radiopacity that is 100 Hounsfield units (HU) or more, beneficially anywhere ranging from 100 HU to 250 HU to 500 HU to 750 HU to 1000 HU to 2500 HU or more.

In some embodiments, the radiopaque crosslinked reaction products break down in-vivo over a period ranging from 2 to 26 weeks.

In some aspects of the present disclosure, systems are provided that are configured to deliver (a) first multifunctional molecule as described hereinabove and (b) a second multifunctional molecule as described hereinabove. The first multifunctional molecule and second multifunctional molecule are comingled such that azide groups of the first multifunctional molecule react and form covalent bonds with the cyclic alkyne groups of the second multifunctional molecule. Such systems can be used to form crosslinked hydrogels, either in vivo or ex vivo.

In some aspects of the present disclosure, systems are provided that comprise (a) a first composition that comprises a first multifunctional molecule as described herein and (b) a second composition that comprises a second multifunctional molecule as described herein.

The first composition may be a first fluid composition or may be first dry composition to which a suitable fluid such as water for injection, saline, etc. can be added to form a first fluid composition. The second composition may independently be a second fluid composition or may be second dry composition to which a suitable fluid such as water for injection, saline, etc. can be added to form a second fluid composition. The first and second compositions may independently be provided in vials, syringes, or other reservoirs.

When the first and second fluid compositions are mixed, covalent bonds are formed between the first multifunctional molecule and the second multifunctional molecule as described above, resulting in a crosslinked reaction product of the first multifunctional molecule and the second multifunctional molecule.

The first and second compositions may further comprise additional agents, including therapeutic agents, imaging agents, colorants, tonicity adjusting agents, suspension agents, wetting agents, and pH adjusting agents.

Examples of therapeutic agents include antithrombotic agents, anticoagulant agents, antiplatelet agents, thrombolytic agents, antiproliferative agents, anti-inflammatory agents, hyperplasia inhibiting agents, anti-restenosis agent, smooth muscle cell inhibitors, antibiotics, antimicrobials, analgesics, anesthetics, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, STING (stimulator of interferon genes) agonists, antimetabolites, alkylating agents, microtubule inhibitors, hormones, hormone antagonists, monoclonal antibodies, antimitotics, immunosuppressive agents, tyrosine and serine/threonine kinases, proteasome inhibitors, matrix metalloproteinase inhibitors, Bcl-2 inhibitors, DNA alkylating agents, spindle poisons, poly (DP-ribose)polymerase (PARP) inhibitors, and combinations thereof.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd(III), Mn(II), Fe(III) and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the hydrogels of the present disclosure, allowing for deep tissue imaging and device marking, for instance, NIR-sensitive nanoparticles such as gold nanoshells, carbon nanotubes (e.g., nanotubes derivatized with hydroxy or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethane (BODIPY) analogs, among others, (e) imageable radioisotopes including 99mTc, 201Th, 51Cr, 67Ga, 68Ga, 111In, 64Cu, 89Zr, 59Fe, 42K, 82Rb, 24Na, 45Ti, 44Sc, 51Cr and 177Lu, among others, and (f) radiocontrast agents such as metallic particles, for example, particles of tantalum, tungsten, rhenium, niobium, molybdenum, and their alloys, which metallic particles may be spherical or non-spherical. Additional examples of radiocontrast agents include non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®).

Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of additional agents further include tonicity adjusting agents such as sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others, suspension agents including various surfactants, wetting agents, and polymers (e.g., albumen, PEO, polyvinyl alcohol, block polymers, etc.), among others, and pH adjusting agents including various buffer solutes.

In various embodiments, a system is provided that includes one or more delivery devices for delivering first and second fluid compositions to a subject. Preferred subjects include mammalian subjects, particularly human subjects.

In some embodiments, the system may include a delivery device that comprises a first reservoir that contains a first fluid composition that comprises a first multifunctional molecule as described above and a second reservoir that contains a second fluid composition that comprises a second multifunctional molecule as described above. When the first and second fluid compositions are mixed, crosslinking occurs between the first multifunctional molecule and the second multifunctional molecule. During operation, the first fluid composition and second fluid composition are dispensed from the first and second reservoirs and combined, whereupon the first and second multifunctional molecules crosslink with one another to form a crosslinked hydrogel.

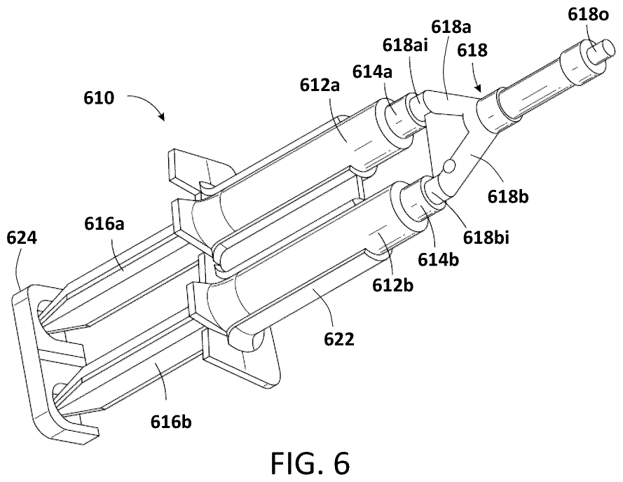

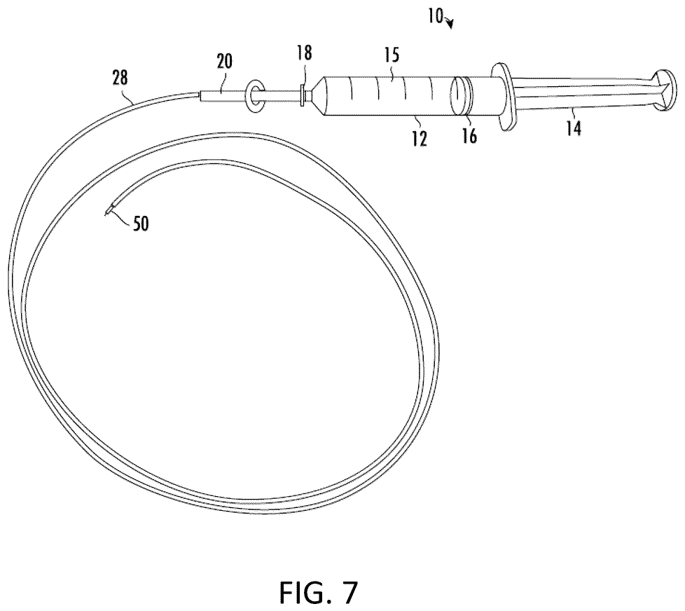

In particular embodiments, and with reference to FIG. 6 the system may include a delivery device 610 that comprises a double-barrel syringe, which includes a first barrel 612a having a first barrel outlet 614a, which first barrel contains the first fluid composition, a first plunger 616a that is movable in the first barrel 612a, a second barrel 612b having a second barrel outlet 614b, which second barrel 612b contains the second fluid composition, and a second plunger 616b that is movable in the second barrel 612b. In some embodiments, the device 610 may further comprise a mixing section 618 having a first mixing section inlet 618ai in fluid communication with the first barrel outlet 614a, a second mixing section inlet 618bi in fluid communication with the second barrel outlet, and a mixing section outlet 618o. Also shown are a syringe holder 622 configured to hold the first and second syringe barrels 612a, 612b, in a fixed relationship and a plunger cap 624 configured to hold the first and second plungers 616a, 616b in a fixed relationship.

In some embodiments, the delivery device may further comprise a cannula or catheter tube that is configured to receive the first and second fluid compositions from the first and second barrels. For example, a cannula or catheter tube may be configured to form a fluid connection with an outlet of a mixing section by attaching the cannula or catheter tube to an outlet of the mixing section, for example, via a suitable fluid connector such as a luer connector.

As another example, the catheter may be a multi-lumen catheter that comprises a first lumen and a second lumen, a proximal end of the first lumen configured to form a fluid connection with the first barrel outlet and a proximal end of the second lumen configured to form a fluid connection with the second barrel outlet. In some embodiments, the multi-lumen catheter may comprise a mixing section having a first mixing section inlet in fluid communication with a distal end of the first lumen, a second mixing section inlet in fluid communication with a distal end of the second lumen, and a mixing section outlet.

During operation, when the first and second plungers are depressed, the first and second fluid compositions are dispensed from the first and second barrels, whereupon the first and second fluid compositions interact and ultimately crosslink to form a crosslinked hydrogel, which is administered onto or into tissue of a subject. For example, the first and second fluid compositions may pass from the first and second barrels, into the mixing section via first and second mixing section inlets, whereupon the first and second fluid compositions are mixed to form an admixture, which admixture exits the mixing section via the mixing section outlet. In some embodiments, a cannula or catheter tube is attached to the mixing section outlet, allowing the admixture to be administered to a subject after passing through the cannula or catheter tube.

As another example, the first fluid composition may pass from the first barrel outlet into a first lumen of a multi-lumen catheter and the second fluid composition may pass from the second barrel outlet into a second lumen of the multi-lumen catheter. In some embodiments the first and second fluid compositions may pass from the first and second lumen into a mixing section at a distal end of the multi-lumen catheter via first and second mixing section inlets, respectively, whereupon the first and second fluid compositions are mixed in the mixing section to form an admixture, which admixture exits the mixing section via the mixing section outlet.

Regardless of the type of device that is used to mix the first and second fluid compositions or how the first and second fluid compositions are mixed, immediately after an admixture of the first and second fluid compositions is formed, the admixture initially may be in a fluid state and can be administered to a subject (e.g., a mammal, particularly, a human) by a variety of techniques. Alternatively, the first and second fluid compositions may be administered to a subject independently and a fluid admixture of the first and second fluid compositions formed in or on the subject. In either approach, a fluid admixture of the first and second fluid compositions is formed and used for various medical procedures.

For example, the first and second fluid compositions or a fluid admixture thereof can be injected to provide spacing between tissues, the first and second fluid compositions or a fluid admixture thereof can be injected (e.g., in the form of blebs) to provide fiducial markers, the first and second fluid compositions or a fluid admixture thereof can be injected for tissue augmentation or regeneration, the first and second fluid compositions or a fluid admixture thereof can be injected as a filler or replacement for soft tissue, the first and second fluid compositions or a fluid admixture thereof can be injected to provide mechanical support for compromised tissue, the first and second fluid compositions or a fluid admixture thereof can be injected as a scaffold, the first and second fluid compositions or a fluid admixture thereof can be injected as an embolic composition, the first and second fluid compositions or a fluid admixture thereof can be injected as lifting agents for internal cyst removal, and/or the first and second fluid compositions or a fluid admixture thereof can be injected as a carrier of therapeutic agents in the treatment of diseases and cancers and the repair and regeneration of tissue, among other uses. The first and second fluid compositions or a fluid admixture thereof can also be injected into a left atrial appendage during a left atrial appendage closure procedure. In some embodiments, the first and second fluid compositions or a fluid admixture thereof may be injected into the left atrial appendage after the introduction of a closure device such as the Watchman® left atrial appendage closure device available from Boston Scientific Corporation.

After administration of the compositions of the present disclosure (either separately as first and second fluid compositions that mix in vivo or as a fluid admixture of the first and second fluid compositions) a crosslinked hydrogel is ultimately formed at the administration location.

After administration, the compositions of the present disclosure can be imaged using a suitable imaging technique such as ultrasound or an x-ray-based imaging technique, such as computerized tomography or X-ray fluoroscopy.

As seen from the above, the compositions of the present disclosure may be used in a variety of medical procedures, including the following, among others: a procedure to implant a fiducial marker comprising a crosslinked product of the first and second compositions, a procedure to implant a tissue regeneration scaffold comprising a crosslinked product of the first and second compositions, a procedure to implant a tissue support comprising a crosslinked product of the first and second compositions, a procedure to implant a tissue bulking agent comprising a crosslinked product of the first and second compositions, a procedure to implant an embolic composition comprising a crosslinked product of the first and second compositions, a procedure to implant a lifting agent comprising a crosslinked product of the first and second compositions, a procedure to introduce a left atrial appendage closure composition comprising a crosslinked product of the first and second compositions, a procedure to implant a therapeutic-agent-containing depot comprising a crosslinked product of the first and second compositions, a tissue augmentation procedure comprising implanting a crosslinked product of the first and second compositions, a procedure to introduce a crosslinked product of the first and second compositions between a first tissue and a second tissue to space the first tissue from the second tissue.

The first and second fluid compositions, fluid admixtures of the first and second fluid compositions, or the crosslinked products of the first and second compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Where formed ex vivo, crosslinked hydrogels may be in any desired form, including a slab, a cylinder, a coating, or a particle. In some embodiments, the crosslinked hydrogel is dried and then granulated into particles of suitable size. Granulating may be by any suitable process, for instance by grinding (including cryogrinding), homogenization, crushing, milling, pounding, or the like. Sieving or other known techniques can be used to classify and fractionate the particles. Crosslinked hydrogel particles formed using the above and other techniques may varying widely in size, for example, having an average size ranging from 50 to 950 microns.

In addition to a crosslinked reaction product as described herein, crosslinked hydrogel compositions in accordance with the present disclosure may further contain additional agents, including therapeutic agents, imaging agents, colorants, tonicity adjusting agents, suspension agents, wetting agents, and pH adjusting agents as described above.

Crosslinked hydrogel compositions in accordance with the present disclosure include injectable fluid suspensions of crosslinked hydrogel particles.

In various embodiments, kits are provided that include one or more delivery devices for delivering the crosslinked hydrogel to a subject. Such systems may include one or more of the following: a syringe barrel, which may or may not contain a crosslinked hydrogel as described herein; a vial, which may or may not contain a crosslinked hydrogel as described here; a needle; a flexible tube (e.g., adapted to fluidly connect the needle to the syringe); and an injectable liquid such as water for injection, normal saline or phosphate buffered saline. Whether supplied in a syringe, vial, or other reservoir, the crosslinked hydrogel may be provided in dry form (e.g., powder form) or in a form that is ready for injection, such as an injectable hydrogel form (e.g., a suspension of crosslinked hydrogel particles).

FIG. 7 illustrates a syringe 10 providing a reservoir for a crosslinked hydrogel composition as discussed above. The syringe 10 may comprise a barrel 12, a plunger 14, and one or more stoppers 16. The barrel 12 may include a Luer adapter (or other suitable adapter/connector), e.g., at the distal end 18 of the barrel 12, for attachment to an injection needle 50 via a flexible catheter 29. The proximal end of the catheter 29 may include a suitable connection 20 for receiving the barrel 12. In other examples, the barrel 12 may be directly coupled to the injection needle 50. The syringe barrel 12 may serve as a reservoir, containing a crosslinked hydrogel composition 15 for injection through the needle 50.

The crosslinked hydrogel compositions described herein can be used for a number of purposes.

For example, crosslinked hydrogel compositions can be injected to provide spacing between tissues, crosslinked hydrogel compositions can be injected (e.g., in the form of blebs) to provide fiducial markers, crosslinked hydrogel compositions can be injected for tissue augmentation or regeneration, crosslinked hydrogel compositions can be injected as a filler or replacement for soft tissue, crosslinked hydrogel compositions can be injected to provide mechanical support for compromised tissue, crosslinked hydrogel compositions be injected as a scaffold, and/or crosslinked hydrogel compositions can be injected as a carrier of therapeutic agents in the treatment of diseases and cancers and the repair and regeneration of tissue, among other uses.

The crosslinked hydrogel compositions of the present disclosure may be used in a variety of medical procedures, including the following, among others: a procedure to implant a fiducial marker comprising a crosslinked hydrogel, a procedure to implant a tissue regeneration scaffold comprising a crosslinked hydrogel, a procedure to implant a tissue support comprising a crosslinked hydrogel, a procedure to implant a tissue bulking agent comprising a crosslinked hydrogel, a procedure to implant a therapeutic-agent-containing depot comprising a crosslinked hydrogel, a tissue augmentation procedure comprising implanting a crosslinked hydrogel, a procedure to introduce a crosslinked hydrogel between a first tissue and a second tissue to space the first tissue from the second tissue.

The crosslinked hydrogel compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

After administration, the crosslinked hydrogel compositions of the present disclosure can be imaged using a suitable imaging technique.

Crosslinked hydrogel compositions in accordance with the present disclosure include lubricious compositions for medical applications, compositions for therapeutic agent release (e.g., by including one or more therapeutic agents in a matrix of the crosslinked hydrogel), and implants (which may be formed ex vivo or in vivo) (e.g., compositions for use as tissue markers, compositions that act as spacers to reduce side effects of off-target radiation therapy, cosmetic compositions, etc.).

It should be understood that this disclosure is, in many respects, only illustrative and that changes may be made in details without exceeding the scope of the disclosure. This may include, to the extent that it is appropriate, the use of any of the features of one embodiment being used in other embodiments.

1. A system for forming a crosslinked reaction product comprising (a) a first composition that comprises a first multifunctional molecule that comprises a plurality of first reactive moieties that comprise azide groups and (b) a second composition that comprises a second multifunctional molecule that comprises a plurality of second reactive moieties that comprise cyclic alkyne groups, wherein at least one of the first and second multifunctional molecules is a reactive multi-arm polymer and wherein at least one of the first and second multifunctional molecules comprises an iodinated core.

2. The system of claim 1, wherein the first reactive moieties further comprise hydrolysable linkages through which the azide groups are linked to a remainder of the first multifunctional molecule.

3. The system of claim 1, wherein the cyclic alkyne groups are cyclooctyne-containing groups.

4. The system of claim 1, wherein the second reactive moieties further comprise hydrolysable linkages through which the cyclic alkyne groups are linked to a remainder of the first multifunctional molecule.

5. The system of claim 1, (a) wherein the first multifunctional molecule is a multi-arm polymer having an iodinated core and a plurality of polymer arms that have azide end groups and (b) wherein the second multifunctional molecule is a multi-arm polymer having a non-iodinated core and a plurality of polymer arms that have cyclic alkyne end groups, a multi-arm polymer having an iodinated core and a plurality of polymer arms that have cyclic alkyne end groups, a non-polymeric molecule having a non-iodinated core and a plurality of pendant reactive moieties that comprise cyclic alkyne groups, or a non-polymeric molecule having an iodinated core and a plurality of pendant reactive moieties that comprise cyclic alkyne groups.

6. The system of claim 1, (a) wherein the first multifunctional molecule is a multi-arm polymer having a non-iodinated core and a plurality of polymer arms that have azide end groups and (b) wherein the second multifunctional molecule is a multi-arm polymer having an iodinated core and a plurality of polymer arms that have cyclic alkyne end groups or a non-polymeric molecule having an iodinated core and a plurality of pendant reactive moieties that comprise cyclic alkyne groups.

7. The system of claim 1, (a) wherein the first multifunctional molecule is a non-polymeric molecule having an iodinated core and a plurality of pendant reactive moieties that comprise azide groups and (b) wherein the second multifunctional molecule is a multi-arm polymer having a non-iodinated core and a plurality of polymer arms that have cyclic alkyne end groups or a multi-arm polymer having an iodinated core and a plurality of polymer arms that have cyclic alkyne end groups.

8. The system of claim 1, (a) wherein the first multifunctional molecule is a non-polymeric molecule having a non-iodinated core and a plurality of pendant reactive moieties that comprise azide groups and (b) wherein the second multifunctional molecule is a multi-arm polymer having an iodinated core and a plurality of polymer arms that have cyclic alkyne end groups.

9. The system of claim 1, wherein the iodinated core comprises one or more iodinated aromatic groups.

10. The system of claim 1, wherein the iodinated core comprises an iodinated polyol residue.

11. The system of claim 1, comprising the first composition in a first container and the second composition in a second container.

12. The system of claim 11, wherein the first and second containers are independently selected from vials and syringes.

13. The system of claim 1, further comprising a delivery device that is configured to deliver the first and second compositions.

14. The system of claim 13, wherein the delivery device comprises a dual barrel syringe.

15. A crosslinked network formed by combining (a) a first multifunctional molecule that comprises a plurality of first reactive moieties that comprise azide groups and (b) a second multifunctional molecule that comprises a plurality of second reactive moieties that comprise cyclic alkyne groups, wherein at least one of the first and second multifunctional molecules is a reactive multi-arm polymer and wherein at least one of the first and second multifunctional molecules comprises an iodinated core.

16. The crosslinked network of claim 15, wherein the crosslinked network is a hydrogel.

17. The crosslinked network of claim 15, wherein the crosslinked network has a radiopacity that is greater than 250 Hounsfield units.

18. The crosslinked network of claim 15, in the form of a suspension of particles of the crosslinked network.

19. The crosslinked network of claim 18, wherein the suspension of particles is provided in a syringe.

20. A method of treatment comprising administering to a subject a mixture that comprises (a) a first multifunctional molecule that comprises a plurality of first reactive moieties that comprise azide groups and (b) a second multifunctional molecule that comprises a plurality of second reactive moieties that comprise cyclic alkyne groups, wherein at least one of the first and second multifunctional molecules is a reactive multi-arm polymer and wherein at least one of the first and second multifunctional molecules comprises an iodinated core.
